(19) United States
(12) Patent Application Publication
(10) Pub. No.: US 2025/0276440 A1
(43) Pub. Date: Sep. 4, 2025

(54) SUPER-ELASTIC EXOSKELETON DEVICE AND METHOD OF USE THEREOF FOR PREVENTION AND MINIMIZATION OF INJURIES TO JOINTS, TENDONS, LIGAMENTS, OR MUSCLES

(71) Applicant: Paul Richard HALLEN, Colleyville, TX (US)

(72) Inventor: Paul Richard HALLEN, Colleyville, TX (US)

(21) Appl. No.: 18/593,278

(22) Filed: Mar. 1, 2024

(51) Int. Cl. B25J 9/00; A61H 1/02

(52) U.S. Cl. CPC B25J 9/0006; A61H 1/024; A61H 1/0244; A61H 1/0277; A61H 1/0281; A61H 1/0285; A61H 1/0292; A61H 2201/165; A61H 2201/169

(57) ABSTRACT

An injury mitigation and prevention support device is provided including flexible support material configured to wrap around a predetermined joint, tendon, ligament, or muscle of a mammal and nitinol elements included in or on the support material, wherein the nitinol elements are configured to be set into an austenite super-elastic phase by a body temperature of the mammal when the support material is wrapped around the joint, tendon, ligament, or muscle and the nitinol elements are located and configured to provide a counteracting force to a force exerted on the joint when the joint, tendon, ligament, or muscle is bent or stretched beyond a normal angle of extension of the joint, tendon, or muscle. As a result, the device acts dynamically and instantaneous with super-elasticity to prevent or minimize acute injury insult, and also acts to continuously to support and reduce fatigue to minimize acute and chronic injury.

### BACKGROUND

Joint, tendon, ligament, or muscle injuries are very common for athletes, workers, and virtually anyone engaged in physical activity. These injuries can happen to any joint including toes, ankles, knees, hips, back, neck, shoulders, elbows, wrists, and fingers and can be debilitating for the person (or animal) suffering from the sprain. Such injuries cause temporary or chronic pain, reduce performance, and, in extreme cases, prevent the person or animal from being able to perform their desired activities at all.

Further, extreme joint instability or hyperextension can result in devastating traumatic injury to ligaments, tendons, and muscles with partial or complete tears. Common examples include damage to the Achilles tendon, Patella tendon, Anterior Cruciate ligament (ACL), Medial Collateral (MCL) ligament, Hamstring, Pectoral, and Calf muscles, etc. Even when the injury is not extreme, repetitive motions in work, athletics, or activities of daily living can cause cumulative stress and strain, inflammation, pain, and instability of joints, ligaments, tendons, and muscles. This can result in temporary or permanent repetitive motion injuries reducing performance or inability to perform at all.

Joint Sprains can happen to any joint including toes, ankles, knees, hips, back, neck, shoulders, elbows, wrists, and fingers and can be debilitating for an athlete or worker causing temporary or chronic pain, reduced performance, or inability to perform at all. Similarly, animals can suffer from joint injuries which impair their ability to perform their activities. For horses this can include ranch horses, racehorses, equestrian/jumping horses, barrel racing horses, rodeo horses, etc. For dogs this can include racing dogs, sledding dogs, herding dogs, etc.

Joint, neck, and back sprains, are common injuries in various sports including, but not limited to, basketball, soccer, football, baseball, softball, handball, volleyball, lacrosse, rugby, Australian Rules Football, gymnastics, wrestling, weight lifting, running, golfing, tennis, pickle ball, racquetball, squash, track and field events, boxing, martial arts, machine racing sports (auto, motorcycle bikes, watercraft) rodeo (bull riding, etc.), water sports (water-skiing, jet-skiing, diving, etc.), winter sports (ice skaters, ice dancers, hockey, snow skiing, snowboarding, snowboard freestyle and racing ski-jumping, cross country skiing, and snow-mobiling, bobsledding, etc.) dancing (ballet, ballroom, modern dance), etc. In fact, joint, tendon and muscle injuries regularly occur in virtually any sport in which there is dynamic human movement including but not limited to running, jumping, lifting, movements causing instability, repetitive motion injury, or hyperextension. Similarly, joint sprains and chronic injuries can also happen in non-athletic professions due to workplace activities requiring excessive or chronic use of joints, which also often lead to repetitive motion injuries.

Prevention of such joint, tendon and muscle injuries has been attempted with limited success through a variety of braces, sleeves, or wrapping with supportive bandage or athletic tapes. All of these previous devices attempt to provide “constraint” or improve stability of joints within the normal range of motion. However, such devices not only provide limited success, but also frequently hinder performance due to bulkiness of the devices.

For example, conventionally, woven fibrous athletic support tape has been used to “wrap” the players to constrain and prevent the ankle roll or to minimize its damage and grade of sprain severity. Despite this precaution, it is not uncommon to see a player on one team or another sprain their ankle in virtually every game. The tape lacks the elasticity to both constrain joints and provide stability to prevent hyperextension of the joint beyond normal range of motion. Further, the tape may loosen during the wearing period due to its relatively poor elasticity or it may deform during use and is unable to return reversibly to its original pre-deformation position or conforming shape. Additionally, the support it does provide is not “instantaneous”, as movement of the foot and ankle initiates deformation in tape followed by constraint.

In summary, lack of support can cause instability and acute injury independent of fatigue. Additionally, lack of support can cause fatigue, stress, and strain which can induce greater instability and lead to both acute and chronic injury. Ideally, a device that exhibits both super-elasticity and instantaneous shape memory could provide continuous stability support to reduce fatigue, stress, strain, and both acute and chronic injury.

### SUMMARY

In an implementation, an exoskeleton device, and a method of use thereof, is provided including flexible support material configured to wrap or fitted around a predetermined joint of a mammal and nitinol elements included in or on the support material, wherein the nitinol elements are configured to be set into an austenite super-elastic phase by a body temperature of the mammal when the support material is wrapped or fitted around the joint, and the nitinol elements are located and configured to provide a continuously dynamic counteracting force to a force exerted on the joint when the joint is bent up to and beyond a normal angle of extension of the joint.

In another implementation of the present disclosure a method is provided of optimizing performance of an exoskeleton device for mitigating and/or preventing injury to a user wearing the exoskeleton device, including arranging nitinol elements comprising at least one of nitinol wires, nitinol tubes and nitinol sheets in and/or on the exoskeleton device in a predetermined arrangement utilizing a design tool configured to perform at least one of finite element analysis, artificial intelligence analysis, and modeling and simulation analysis to integrate data, stored in a database accessible to the design tool, regarding at least one of human and/or animal anatomy, kinesiology, and ergonomics.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

The drawing figures depict one or more implementations in accord with the present teachings, by way of example only, not by way of limitation. In the figures, like reference numerals refer to the same or similar elements. Furthermore, it should be understood that the drawings are not necessarily to scale.

FIG. 1 illustrates an example of an example of an ankle injury which the device of the present disclosure can be utilized for in order to mitigate or prevent the injury, in accordance with an aspect of the present disclosure.

FIG. 2 illustrates degrees of the ankle injury shown in FIG. 1.

FIG. 3 is a simplified schematic drawing showing the construction of an exoskeleton ankle wrap device that can be used to mitigate or prevent the ankle injury shown in FIG. 1, in accordance with one aspect of the present disclosure.

FIG. 4 shows an alternative implementation of a support ankle wrap on an ankle in which nitinol wires, tubes or sheets are sewn into (or located on an outer or inner side of) the support ankle wrap to mitigate or prevent the ankle injury shown in FIG. 1, in accordance with aspects of the present disclosure.

FIG. 5 is a simplified schematic drawing showing the construction of an alternative implementation of an ankle wrap device that can be used to mitigate or present the ankle injury shown in FIG. 1, in accordance with one aspect of the present disclosure.

FIG. 6 is a simplified schematic drawing showing the construction of an implementation of an exoskeleton wrist device that can be used to mitigate or prevent a wrist injury caused by hyperextension similar to the ankle injury shown in FIG. 1, in accordance with one aspect of the present disclosure.

FIG. 7 shows an exoskeleton support wrist wrap on a wrist in which nitinol wires, tubes or sheets are sewn into or located on an outer (upper) side of the support wrist wrap to mitigate or prevent a wrist injury caused by hyperextension of the wrist in a downward direction (i.e., on an inner (lower) side of the wrist opposite to the outer side that the nitinol wires, tubes or sheets are located on), in accordance with aspects of the present disclosure.

FIG. 8 shows an exoskeleton support wrist wrap on a wrist in which nitinol wires, tubes or sheets are sewn into or located on an inner (lower) side of the support wrist wrap to mitigate or prevent a wrist injury caused by hyperextension of the wrist in an upward direction, in accordance with aspects of the present disclosure.

FIG. 9 shows examples of patterns which can be used for a sheet of nitinol material, particularly for wearable designs, in accordance with aspects of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The present disclosure generally relates to use of super-elastic materials and/or shape memory materials for use in providing or enhancing joint stability, constraint, or support to mitigate and/or prevent athletic or workplace injuries relating to joint instability, fatigue, repetitive motion injury, and/or joint hyperextension. These materials can effectively act as a protective exoskeleton (e.g., a wearable device, structure or article of clothing designed to help prevent injury) with unprecedented step changes in performance for one wearing the device. It is noted that this protective exoskeleton can be in the form of an athletic support tape, a wrap, a sleeve, a brace, a splint or an article of protective clothing.

More particularly, the present disclosure utilizes the metal alloy of nickel and titanium (NiTi), known as Nitinol, to provide protection for joints by providing targeted continuously dynamic counteracting forces to forces exerted on the joints, ligaments, tendons, and muscles during activity. As will be discussed below, the relative percentages of Ni and Ti can be tailored to provide the desired response to temperature to ensure that the necessary counteracting forces are applied during activities of the user. For example, nitinol 55 (having a 55/45 ratio of Ni to Ti) and nitinol 60 (having a 60/40 ratio of Ni to Ti) are two such alloys which can be used in the present disclosure. In this regard, the ration of Ni to Ti determines when the temperature at which the nitinol enters into it super-elastic austenite phase, which is the phase that is used in the present disclosure to generate the desired continuously dynamic counteracting forces to forces on the joint that can lead to injury of ligaments, tendons, and muscles, etc.

Nitinol is used in the present disclosure because it has properties that provide unique capabilities to allow generation of the desired injury mitigating/preventing counteracting forces. Specifically, above its transformation temperature (in the austenite phase) nitinol behaves super-elastically and instantaneously. In other words, when nitinol is stretched in the austenite phase, above the transformation temperature, it will immediately attempt to snap back to its original position. On the other hand, when the nitinol is below the transformation temperature (in the martensite phase) it exhibits only a shape memory effect. In other words, when nitinol is in the martensite phase, below the transformation temperature, when stretched it will stay in the stretched position without super-elastic counteracting support forces

Additionally, another important nitinol material characteristic for the present disclosure is that nitinol can uniquely deform 10 to 30 times more than ordinary metals and still return to its original shape rapidly when it is in its austenite phase. It is also important to note that the transformation is both reversible bi-directionally and instantaneous. In this regard, it is noted that nitinol metals have different moduli of elasticity and yield strengths in their 2 different phases: e.g., in the martensite (lower temperature) phase, the modulus of elasticity ranges from 28 to 40 GPa and the yield strength ranges from 70 to 140 MPa whereas, in the austenite (higher temperature) phase, the modulus of elasticity ranges from 75 to 83 GPa and the yield strength ranges from 195 to 690 MPa.

Nitinol typically transitions to its super-elastic phase or state at temperatures around 70° F. to 95° F. (21° C. to 35° C.). This temperature range is where the shape memory alloy exhibits its unique super-elastic and shape memory properties, allowing it to recover its original shape after being deformed. This temperature range is well suited to the applications of athletes, workers, and to the broader public in their daily activities of living.

In summary, nitinol's properties allow for free deformation but with instantaneous and super-elastic support and joint stability. In the nitinol integrated athletic support tape embodiments discussed below, the wrapping techniques are identical to standard tape. Uniquely, the nitinol wire, tubes, or sheet temperature is elevated, in accordance with the present disclosure, due to the heat from the human skin during play. This temperature increase shifts the nitinol fibers, tubes or sheets from the martensitic shape memory phase into the austenitic super-elastic phase. As the joint extends (for example, in an ankle roll), the nitinol can deform but super-elastically and instantaneously contracts to resist and constrain nitinol deformation and return to its original shape memory martensitic form. These unique properties provide far superior joint support, constraint, and stability protection against hyperextension and associated tissue damage than is possible with conventional athletic support tapes, wraps, sleeves, supports, braces, etc.

This technology represents a technological breakthrough in joint support and stability and has the potential to reduce injury due to hyperextension dramatically. Arrangement of wire or tube patterns can be designed to mitigate hyperextension of more than one injury type in a particular application. For example, basketball players could be protected from inversion and aversion ankle sprains with a “diamond pattern” of nitinol wires or tubes to counteract complex extension movement in addition to Achilles tendon injuries by linear wires or tubes running axially to the tendon. Similarly, a nitinol sheet could be used to prevent this devastating injury.

Although the descriptions herein illustrate the use of nitinol wires, tubes or sheets in an exoskeleton protective device, it is noted that a plurality of permutations of nitinol elements could be used in the same device to tailor the device for specific types of joint, ligament, tendon and muscle protection. In other words, a single exoskeleton protective device could have a complex combination of nitinol elements. For example, the device could have two or more nitinol wires or tubes with different outer wire diameters (OD), or wires and sheets with different thicknesses and patterns, or a mix of two or more of wires, tubes and sheets in the same device, or a combination of any of these.

As will be discussed in detail below, prevention of joint/tissue injury is facilitated by instantaneous and super-elastic shape memory response (e.g., super-elastic deformation and reformation) to dynamic plastic deformation of device. The key attributes resulting in injury prevention are stability maximization and fatigue minimization. The devices and methods described herein will continuously, dynamically, and instantaneously respond to plastic deformations and thereby provide unsurpassed stability of the joints, ligaments, tendons, and muscles. This, in turn, reduces fatigue on the protected joints which is critical because fatigue can increase the probability of injury. When a dramatic plastic deformation occurs the instantaneous and proportionate counter-force response can blunt traumatic injury.

An example of the importance of instability and fatigue being causative to injury is that studies show that thoroughbred racehorses are prone to break bones in their front legs below the third metatarsal near the sesamoid bone and do so 70% of the time in the third and fourth turns and final straight of a race, which is the time when the horses are most fatigued. Only 30% of these injuries occur to racehorses in the first and second turns and backstretch (at the beginning of a race). After an injury the device can help prevent reoccurrence of the same injury, again by providing maximum joint stability and minimum joint fatigue.

As will be appreciated from the following discussion, the present disclosure provides arrangements for optimizing performance of an exoskeleton device for mitigating and/or preventing injury to a user wearing the exoskeleton device, including arranging nitinol elements including at least one of nitinol wires, nitinol tubes and nitinol sheets in and/or on the exoskeleton device in a predetermined arrangement utilizing a design tool configured to perform at least one of finite element analysis, artificial intelligence analysis, and modeling and simulation analysis to integrate data, stored in a database accessible to the design tool, regarding at least one of human and/or animal anatomy, kinesiology, and ergonomics.

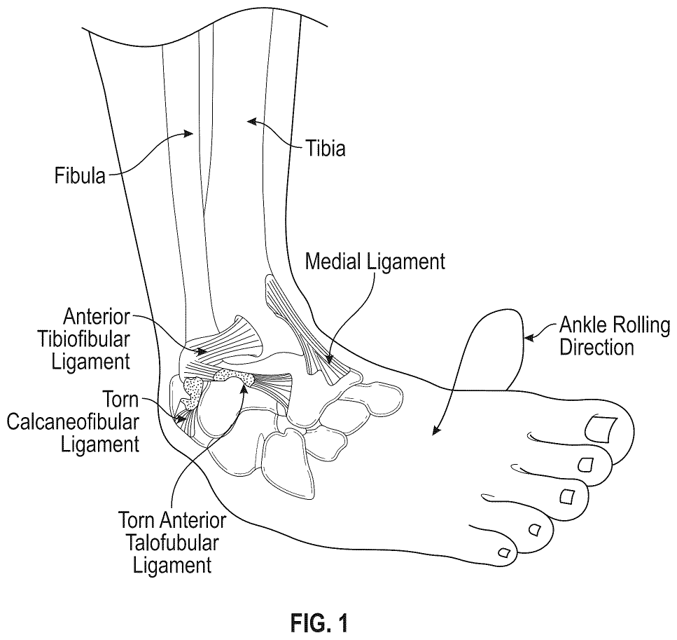

FIG. 1 illustrates an example of an example of an ankle injury, as one example of an injury which the device and method of the present disclosure can mitigate or prevent. This implementation is used to elucidate the use case and enabling the unique protective mechanism of action (MOA) of the device and method of the present disclosure, but, as will be discussed below, this same MOA can be used to protect a wide variety of joints, and each product application implementation relies on the same fundamental principles discussed with regard to this example of an ankle injury.

The sport of basketball has the most joint sprains of any competitive sport, and sprained ankles are the predominant injury in the sport. The combination of highly dynamic running and jumping (In the NBA on average players elevate 28 inches above the floor and at highest 48 inches), rubber soled shoes designed to grip the floor, and athletes with large feet is the perfect combination for frequent ankle injuries. However, such ankle injuries can occur in many other sports as well, especially those such as tennis, squash, racquet ball, handball, etc., that involve jumping and hard surfaces.

The so-called “ankle roll” shown in FIG. 1 is the most common cause of ankle sprain. Using basketball as an example, frequently an offensive basketball player jumps to shoot or rebound the basketball. As the player lands from a nominal height of 24 to 48 inches above the floor, the offensive player may land on the basketball shoe of another player, usually the opposing defender who, due to relative lateral movement in defense, now inadvertently occupies the offensive player's landing space. In this situation, one foot of the offensive shooter lands on the irregular surface of the top of the shoe of the defender. Unlike the accommodating and expected flat wood surface of the court, the defender's foot causes the ankle of the offensive shooter to roll laterally outward within the shoe, thereby hyperextending the ankle of the offensive shooter. This hyperextension is unexpected by the landing player since they are expecting a flat surface below and are generally not looking at their feet to anticipate the ankle roll.

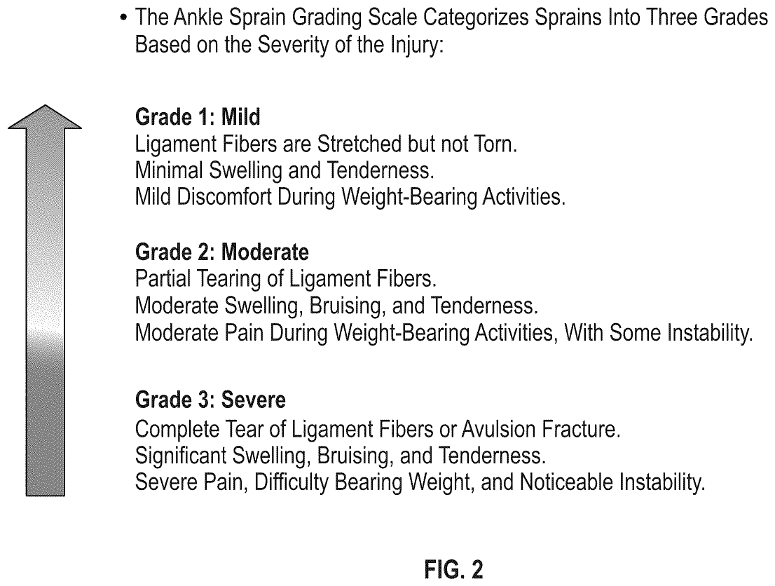

FIG. 1 shows an example of what can happen to the landing player's ankle during an ankle roll. During the hyperextension upon landing soft tissues may incur damage and ligaments may be partially or completely torn. FIG. 1 shows, for example, a situation where the ankle roll causes tearing of the calcaneofibular ligament and the anterior talofibular ligament. These ankle joint sprains may be graded from minor Grade 1 to severe Grade 3 complete ligament tears, as shown in FIG. 2.

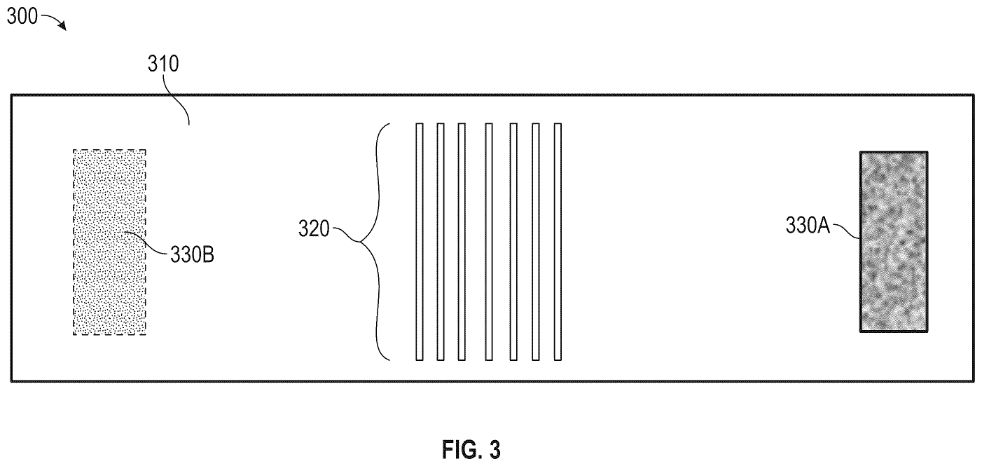

FIG. 3 is a simplified schematic drawing showing the construction of an ankle wrap device 300 with a flexible support material 310 (such as conventional athletic support tape) configured to wrap around an ankle joint. Nitinol elements 320 (e.g., wires or tubes) are integrated and/or woven into the flexible support material 310 (or attached to an upper or lower surface thereof) to mitigate or prevent the ankle injury shown in FIG. 1. Velcro™ pads 330A and 330B are provided on opposite surfaces of the flexible support material 310 so that the flexible support material can be wrapped around the ankle and secured thereto with the Velcro™ pads 330A and 330B.

In accordance with the principles of the present disclosure, the flexible support material 310 should be wrapped around the ankle so that the nitinol elements 320 will cover at least the outer portion of the ankle (i.e., the portion of the ankle which will be stretched during an ankle roll). In this way, when the ankle roll shown in FIG. 1 occurs, the nitinol elements 320 will stretch with the ankle roll but then (assuming that the nitinol elements 320 are in their super-elastic austenite phase due to the body temperature of the user) instantly retract to provide a strong protective counteracting force against the force of the ankle roll. In other words, as the nitinol elements 320 begin to stretch due to the ankle roll, they will instantly try to contract to their original length, thereby providing the desired counteracting force to the forces being generated by the ankle roll. This counteracting force will effectively try to maintain the ankle in its original position to avoid damaging hyperextension of the ankle joint. Depending on the force exerted by the ankle roll, and the strength of the counteractive force exerted by the retracting nitinol elements 320, the damage caused by the ankle roll can be prevented or, at a minimum, significantly reduced. Current conventional taping of ankles provides mobility reducing constraint which has a protective effect, but at the expense of significantly reducing mobility. The super-elastic exoskeleton device of the present disclosure, on the other hand, provides continuous dynamic support and instantaneous response to damaging deformation forces with much less constraint of the user's mobility than current conventional devices. Note that the nitinol material can also be integrated into athletic support tape and combine the containment capability of tape and the instantaneous super-elasticity derived support, stability, and fatigue reduction.

As noted above, and as shown in FIG. 3, the nitinol elements 320 should be located in or on the flexible support material 310 to be in an appropriate position to exert a counteracting force to the force (such as an ankle roll) that can cause injury. This location for the nitinol elements 320 in or on the flexible support material 310 will be different depending on what type of joint is being protected. For example, the location of the nitinol elements 320 will be different for protecting a knee, shoulder, back, neck, etc. will be tailored to ensure that the counteracting forces will be applied when the joint in question is being bent in a direction that can cause injury to the joint.

The location of the nitinol elements 320 in FIG. 3 is targeted to specifically protect against an outward ankle roll. This is the most common type of ankle injury, and targeting this area for protection allows for protecting against this common injury while minimizing cost, manufacturing steps and weight of the ankle wrap device 300. However, in alternative implementations, additional nitinol elements 320, spaced apart from the nitinol elements 320 shown in FIG. 3, could be provided to protect against an inward ankle roll, for example. And, of course, the nitinol elements 320 could be provided completely along the length of the flexible support material 310, if desired, to provide protection completely around the ankle.

As noted above, it is important that the nitinol elements 320 be constructed so that they will be in the super-elastic austenite phase during the user activity for which protection is necessary. This involves selecting the ratios of Ni/Ti metals, the wire/tube diameters of the nitinol and the placement of the nitinol elements 320 in the flexible support material so that the nitinol elements 320 will be sufficiently heated by the body heat of the user to exceed the transformation temperature of the elements 320 to enter the austenite phase and behave super-elastically. This super-elastic behavior is the basis of the present disclosure for providing the protective counteracting force against forces, such as ankle rolling, that can cause joint damage.

The super-elastic nitinol athletic wrap device 300 is typically worn under an athlete's sock (for example, as a wrapping support tape). The sock can be designed optimally to capture the heat to help achieve and maintain the activation temperature. Once the woven nitinol tape fully reaches its phase transformation temperature, it provides the necessary super-elastic support. During moments of ankle stress, the nitinol fibers act to prevent excessive ankle movement, potentially reducing the severity of sprains (e.g., as shown in FIG. 2) from Severe to Moderate, Moderate to Mild, or even preventing them.

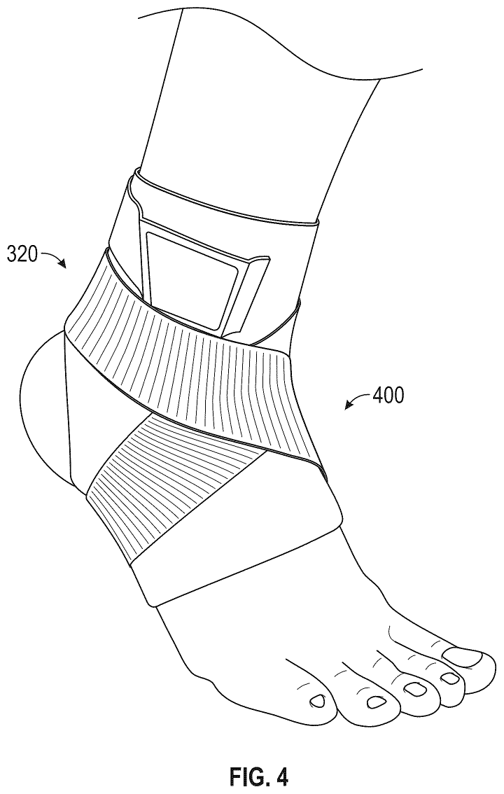

FIG. 4 shows an alternative implementation of a support ankle wrap 400 on an ankle in which nitinol wires, tubes or sheets are sewn into (or located on an outer or inner side of) the support ankle wrap to mitigate or prevent the ankle injury shown in FIG. 1. This ankle wrap 400 can be a commercially available ankle wrap which has been modified in accordance with the present disclosure to include nitinol wires, tubes or sheets incorporated therein or thereon to provide the same protective effects noted above for the FIG. 3 implementation.

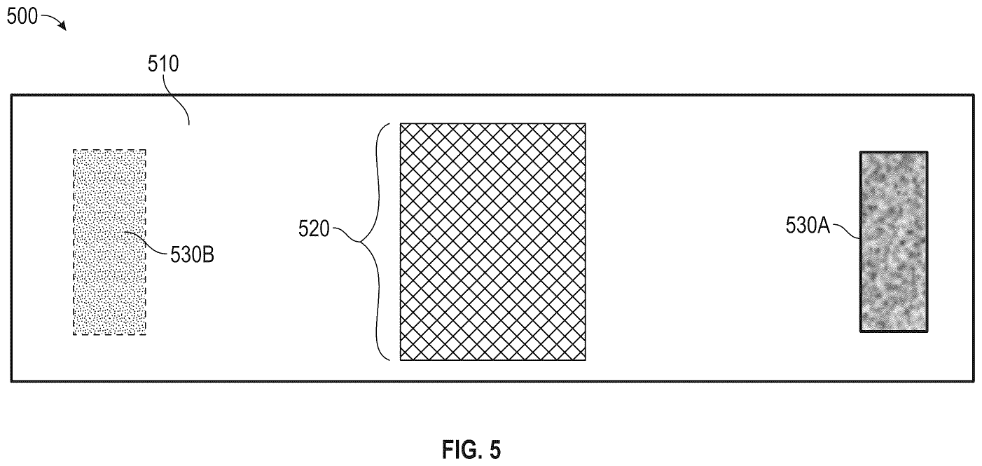

FIG. 5 is a simplified schematic drawing showing the construction of another alternative implementation of an ankle wrap device 500 that can be used to mitigate or prevent the ankle injury shown in FIG. 1. The wrap device 500 is similar to the wrap device 300 of FIG. 3 in terms of being formed with a flexible wrap material 510 configured to wrap around an ankle, and Velcro™ pads 530A and 530B. However, in place of the nitinol elements 320 of FIG. 3, which can be parallel wires of tubes, the implementation of FIG. 5 uses a nitinol sheet 520 to create the desired counteractive force to the ankle rolling forces. This nitinol sheet 520 can be woven into the flexible wrap material 510 or attached on an inner or outer side of the wrap material 510. In any case, similar to the nitinol elements 320 of FIG. 3, it is necessary for the nitinol sheet 520 to be located in or on the wrap material 510 in a location that will allow the nitinol sheet 520 to be in the austenite phase and to be over the outer portion of the ankle to counteract the ankle roll.

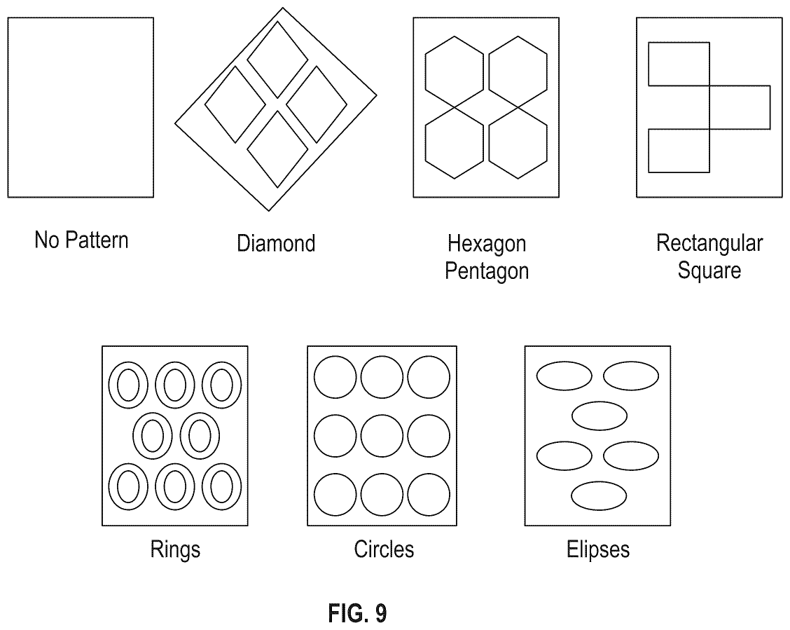

The nitinol sheet 520 can take a number of forms. For example, the nitinol sheet 520 can be made by weaving a number of nitinol wires together to form a nitinol metal fabric sheet. On the other hand, the nitinol sheet 520 can simply be a thin sheet of solid nitinol material. In addition, a number of patterns can be formed in the nitinol sheet, whether it is woven or a solid sheet, as shown in FIG. 9. Specifically, as shown in FIG. 9, the shapes can include, but are not limited to, diamonds, hexagons, pentagons, rectangles, rings, circles, and ellipses. These patterns can be either cut all the way through the nitinol sheet 520, or cut in a relief manner part way through the sheet. In any case, these patterns (as opposed to a simple “no pattern” flat sheet) can be useful for providing greater flexibility for the nitinol sheet during movement of the user and can also help with ventilation to prevent a buildup of heat under the sheet. Also, as shown below in Table 1, using a nitinol sheet 520 instead of elongated wires or tubes can provide greater strength than wires or tubes in some applications and greater, faster, and/or more stable heat transfer.

Regardless of whether nitinol wires or tubes are used, as shown in FIG. 3, or a nitinol sheet is used, as shown in FIG. 5, (or a combination of these nitinol elements) the characteristics of the nitinol, and the characteristics of the wires, tubes or sheets, may be tailored in order to provide the desired counteracting force for the particular joint being protected. For example, as noted above, the nickel/titanium ratios of the nitinol are an important factor and determining the genetic activation temperature points (that is, the transformation temperature when the nitinol changes from its martensite state to its austenite state). The location of the nitinol wires, tubes and sheets in the flexible support material is also important with regard to the transformation temperature point in terms of ensuring that body heat from the user reaches the nitinol material in order to activate it into the super-elastic austenite state.

Similarly, as shown in Table 1 below, the characteristics of the wires, tubes and sheets in terms of number, size, and thickness is important in determining how much counteracting force will be generated in response to a potentially injurious force on the joint.

### TABLE 1

Solid Wires: A plurality of gauge sizes and Nickel:Titanium ratios
Hollow Tubes: Plurality of gauge sizes (Plurality of: Outside diameter, Inside diameter, and associated wall thickness) and Nickel:Titanium ratios for different performance characteristics and Austenetic activation temperature points.
Flat Sheets with and without Patterns or Relief Cut elements
   Geometric Patterns including but not limited to a plurality of shapes
   Plurality of sheet thicknesses
   Nickel: Titanium ratios
   Patterns can be LASER cut from sheets (existing process) with the sheet providing greater strength than wires or tubes in some applications and greater, faster, and/or more stable heat transfer while increasing flexibility and deformation than a sheet with “No Pattern”.

For example, referring to FIG. 3, the number and gauge of the nitinol wires or tubes 320 will determine the magnitude of the counteracting force generated when the joint is bent. Similarly, the size and thickness of the nitinol sheet 520 in FIG. 5 will determine the amount of counteracting force that is generated. In accordance with aspects of the present disclosure, these parameters (e.g., number of wires or tubes, size of the sheet, thicknesses of wires, tubes and sheets, location of the wires, tubes and sheets, etc.) may be tailored to provide the amount of counteracting force necessary to protect the joint while not being so large as to hamper the bending of the joint to the point of not allowing the user to perform the desired motion. This tailoring of the parameters and location of the nitinol elements can be performed by measuring the forces generated on a joint when it is bent, and then adjusting the parameters and location of the nitinol elements (e.g., wires, tubes or sheets) to provide the appropriate amount of counteracting force to the measured force to prevent injury while still allowing the user to perform the activity.

In FIGS. 3 and 5, the nitinol elements 320 and sheets 520, respectively, are shown in a location that will allow them to cover the outer portion of the user's ankle when the device is wrapped around the ankle. As noted above, it is most desirable for the nitinol elements 320 and sheets 520 to be located so that the nitinol will be stretched as the joint is bent. However, in accordance with an alternative implementation of the present disclosure, additional nitinol elements or sheets could be provided at different locations on the flexible support material to provide protection from other potential injuries as well. For example, in the implementation of FIG. 3, the nitinol elements 320 are arranged to protect against ankle rolling damage in an outward direction of the ankle (which is by far the most common ankle injury). However, nitinol elements 320 could also be located to cover other parts of the ankle that might also sustain injury, such as the inner side of the ankle or the top of the ankle, if subjected to unnatural or excessive bending.

The above discussion has used ankle injuries as an exemplary use of the devices and methods of the present disclosure. However, as also noted above, the present disclosure is directed to preventing, or at least mitigating, joint injuries for all types of joints, both in humans and animals. In each instance, the composition of the nitinol, the characteristics of the nitinol elements or sheets, and the locations of these elements will need to be tailored to be appropriate for the joint in question. For example, the compositions, dimensions and locations of the nitinol elements or sheets to protect a finger joint will be different than those necessary to protect an ankle, knee or shoulder joint.

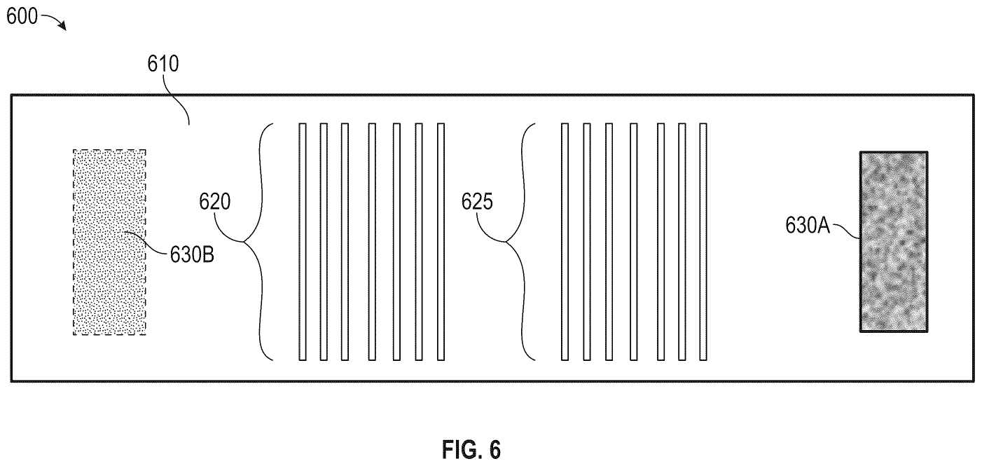

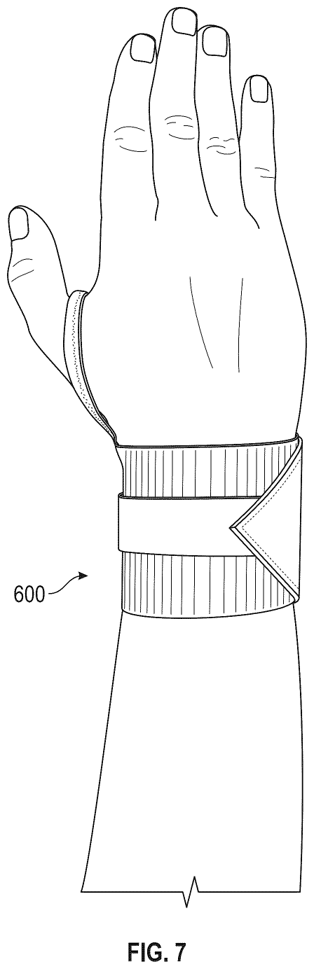

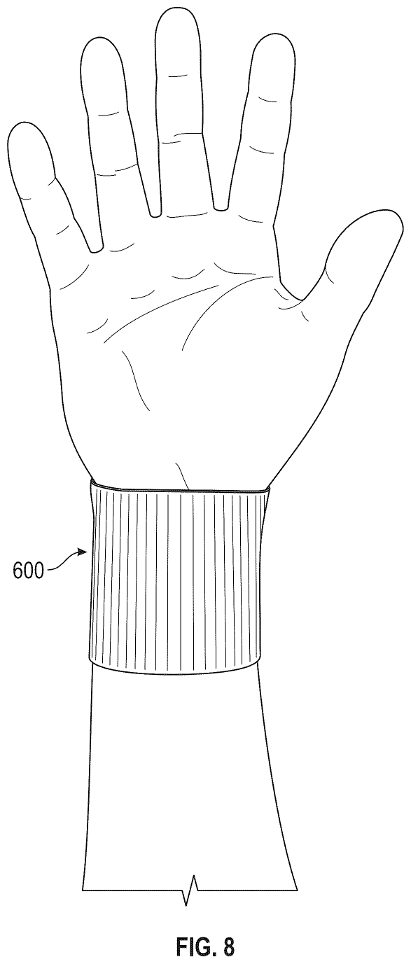

FIGS. 6-8 show an example of a protective wristband 600 utilizing the principles noted above for producing counteractive forces to protect a wrist, regardless of whether the is bent in an upward position or a downward position. To this end, the protective wristband 600 includes the flexible support material 610 and Velcro™ pads 630A and 630B (similar to the implementation shown in FIGS. 3 and 5) and a set of nitinol elements 620 and 625. When the flexible support material 610 is wrapped around a user's wrist, the nitinol elements 620 are located to cover the upper (top) part of the wrist (as shown in FIG. 7) and the nitinol elements 625 are located to cover the lower (bottom) part of the wrist (as shown in FIG. 8). In this case, regardless of whether the wrist is being bent excessively in an upper direction or a lower direction, a counteracting force is generated be either the nitinol elements 620 and 625 (for example, nitinol wires or tubes) to protect the wrist joint from injury.

The above description illustrates principles of the present disclosure with particular reference to preventing/mitigating ankle and wrist injuries. The following description provides additional details regarding preventing/mitigating such ankle and wrist injuries in humans as well as examples of numerous other applications of use of the devices and methods of the present disclosure for other joints in both humans and animals.

The following implementation categories can be designed to be either single-use disposable or reusable devices. Key fields of use for the above-described devices and methods in accordance with the present disclosure include but are not limited to:
Human Athletics
Animal Athletics
Occupational/Labor activities
Human Activities of Daily Living
Active Clothing Integration
Specialty Protective Applications: Police, Fire-fighters, Military, Astronaut, Pilots, Elderly, Handicapped, Automotive, Motorcycle, Skateboard, Ski, Snowboard, Water Ski, Jet Ski

These key fields will be discussed below.

In the field of human athletics, this can include either single-use or reusable: Athletic support tape with integrated and/or woven nitinol wires, tubes or sheets; Athletic support sleeves with integrated and/or woven nitinol wires, tubes or sheets; and Athletic support braces and splints with integrated and/or woven nitinol wires, tubes or sheets. The term human athletics includes, but is not limited to, basketball, soccer, football, baseball, volleyball, lacrosse, rugby, gymnastics, wrestling, weight-lifting, running, golfing, tennis, pickle ball, racquetball, squash, track and field events, boxing, martial arts, machine racing sports (auto, motorcycle bikes, watercraft) rodeo (bull riding, etc.), water sports (water-skiing, jet-skiing, diving, etc.), winter sports (ice skaters, ice dancers, hockey, snow skiing, snowboarding, snow-mobiling, bobsled) dancing (Ballet, ballroom, modern dance, hiking, climbing (mountain, rock) etc. Some of the common injuries which the devices and methods of the present disclosure can help to prevent/mitigate include pitcher's elbow and tennis elbow.

In the field of animal athletics, athletic support tapes, sleeves, braces and sprints similar to those used for human athletes discussed above can be used. Such animal athletics includes, but is not limited to, race horses, rodeo horses, work horses, carriage horses, race dogs, herding dogs, etc. Race horses in particular are prone to ankle and leg injuries. The impact is severe in that the outcome is frequently that the animal must be euthanized. These horses are frequently valued in the millions of dollars for their racing value and breeding value. The solution to prevent these ankle and leg injuries is fundamentally similar to the human examples provided within this disclosure. Nitinol woven and/or integrated into flexible support tapes, sleeves, braces, or splints would provide joint stabilization and prevent/minimize joint damage and/or broken bones.

In the field of occupational and labor activities, flexible support tapes, sleeves, braces and sprints similar to those used for human athletes discussed above can be used. Occupational/Labor activities Included but not limited to: Manufacturing; Agricultural; Industrial; Construction; Delivery, and Warehousing. By providing a counteracting force to stressful joint movements, the devices and methods of the present disclosure can counteract the damaging effects of repetitive motions frequently required in occupational and labor activities. For example, the device's stability can reduce fatigue in chronic situations such as repetitive motion injury in an industrial job to the wrist induced by using a tool repeatedly (torquing rotationally with a screwdriver, squeezing pliers, closing scissors etc.).

Other non-manufacturing and non-athletic professionals are also susceptible to repetitive motion injuries and would benefit from the use of protective devices made using the principles disclosed herein. These professions include, but are not limited to, musicians, chefs/cooks, surgeons, dentists, dental hygienists, etc. For example, musicians who play string instruments, including guitarists, violinists, violists, cellists, bass players, etc. frequently suffer from repetitive stress injuries to their elbows, wrists and fingers. Similarly, chefs and cooks tend to have wrist and elbow issues from cooking techniques such as whisking and stirring. Surgeons, dentists and hygienists, as well as echocardiographers, frequently have problems with their lower arms, wrists and hands. Surgeons and dentists have use microscopes in their work also have neck, shoulder and back issues. People who do a large amount of typing also very frequently suffer from wrist injuries, such as the well-known carpal tunnel syndrome. These are just a few examples of the many areas which could benefit from the devices and methods disclosed herein.

In normal daily living human activities, similar support tapes, sleeves, braces and sprints similar to those used for human athletes discussed above can be used. Such activities include housework and yardwork, where joint injuries often occur.

In the field of active clothing integration, shirts, pants, undergarments, socks, shoes and gloves are examples of active clothing in which nitinol wires, tubes or sheets can be woven into or layered on. This integration of nitinol wires, tubes or sheets into clothing can be very useful to provide all-day support, stability and protection for chronic pain issues. It can also be very useful to help prevent falls, particularly in elderly people by providing supporting forces at ankle, knee and hip joints to prevent buckling of these joints during walking. Prevention of such falls is crucial to prevent the hip fractures and back, knee, head, neck, hand and wrist injuries that these falls frequently cause. Hip fractures are notorious for cause of premature death in just a few years post injury.

In the field of special protective applications, flexible support tapes, sleeves, braces and sprints similar to those used for human athletes discussed above can be used. These specialty protective applications include but not limited to injury prevention in high-risk activity categories such as police, fire-fighters, emergency medical personnel, rescue workers, military, astronauts, pilots, boating/marine, automotive workers, motorcycle riders, elderly people, handicapped people, etc.

As noted above, the principles of the present disclosure regarding the use of nitinol wires, tubes and sheets can be applied to conventional athletic tape. Regarding this, it is noted that the teachings of the present disclosure can be used without hindering the use of the three most popular and effective athletic tape application techniques as follows:

### Stirrup Technique:

Start by applying an anchor strip above the ankle bone on the inside of your foot.
Bring the tape under your arch and wrap it around the back of your heel, crossing over the anchor strip.
Continue wrapping the tape up and around your ankle in a figure-eight pattern, covering the sides and supporting the ankle.
Finish by securing the tape with another anchor strip on the outside of your foot.

### Figure-Eight Technique:

Begin by applying an anchor strip above the ankle bone on the inside of your foot.
Cross the tape over the front of your foot and wrap it around your ankle.
Move the tape diagonally across the back of your heel, forming a figure-eight pattern.
Continue the figure-eight pattern, alternating between the front and back of your ankle, until it is adequately supported.
Secure the tape with an anchor strip on the outside of your foot.

### Basketweave Technique:

Start with an anchor strip above the ankle bone on the inside of your foot.
Apply a second anchor strip below the ankle bone on the outside of your foot.
Apply two strips diagonally from the inside anchor strip to the outside anchor strip, forming an ‘X’ pattern.
Repeat this process, creating a basketweave pattern by alternating strips from inside to outside and vice versa.
Wrapping the tape snugly but not too tight, will provide support without cutting off circulation. Ensure the tape adheres well to the skin helps assure effective support during athletic activities.

In addition to use of the devices and methods of the present disclosure with athletic support tape, these devices and methods can also be used in advanced joint sleeves with nitinol wires, tubes or sheets. Traditional compression fit joint sleeves may be fortified with nitinol wires, tubes or sheets designed with a plurality of diameters and wire patterns depending on the joint application requirements and performance requirements. Traditional compression fit joint sleeves are frequently worn for support over knees, elbows, ankles, and fingers to protect previous moderately injured joints or to prevent primary hyperextension. These joint sleeves are generally somewhat elastic and stretch to conform to the individuals anatomy and are sized to provide an interference fit snuggly or slightly compress the anatomy. These conventional compression fit joint sleeves offer only modest support but have the advantage of not being bulky or cumbersome.

In accordance with the principles of the present disclosure, compression joint sleeves similarly designed to conventional sleeves but with the addition of integrated nitinol wires, tubes or sheets maintain the non-bulky advantages of current sleeves but also provide super-elastic support similar to that described above. The snug fit to the human skin and sleeve material of such compression fit joint sleeves readily elevates the temperature of the nitinol wires, tubes or sheets to the austenitic super-elastic phase. Therefore, the modified compression fit joint sleeves (with the nitinol elements) will provide the desirable counteracting force to prevent/mitigate injury, as described above.

Similarly, in addition to use of the devices and methods of the present disclosure with athletic support tape, these devices and methods can also be used in advanced braces, splints and casts with nitinol wires, tubes or sheets. Traditional knee, neck, wrist, or back braces, splints and casts may be fortified, in accordance with the present disclosure, with nitinol wires, tubes of sheets (designed with a plurality of diameters and/or wire patterns) and/or structural nitinol elements in a plurality of thickness, shapes, and functional mechanisms (hinges, levers, struts etc.) depending on the joint application requirements and performance requirements. Similarly, broken bones may have less bulky super-elastic reinforced casts for superior support while healing. Typically, braces are worn to prevent injury, reduce the probability of re-injury, or reduce fatigue or inflammation due to overuse in daily activities, workplace activities, or in athletic endeavors. Integration of nitinol wires and/or structural elements and/or functional mechanisms in accordance with the present disclosure can reduce the bulkiness of current braces while simultaneously providing far superior super-elastic support. The close proximity of the braces to the human skin serves to elevate the temperature of nitinol elements above the transformation temperature into the austenitic phase providing super-elastic support.

While various embodiments have been described, the description is intended to be exemplary, rather than limiting, and it is understood that many more embodiments and implementations are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made within the scope of the attached claims.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various examples for the purpose of streamlining the disclosure. This method of disclosure is not to be interpreted as reflecting an intention that the claims require more features than are expressly recited in each claim. Rather, as the following claims reflect, inventive subject matter lies in less than all features of a single disclosed example. Thus, the following claims are hereby incorporated into the Detailed Description, with each claim standing on its own as a separately claimed subject matter.

What is claimed is:

1. An injury mitigation and prevention exoskeleton support device, comprising:
flexible support material configured to wrap around a predetermined joint, tendon, ligament, or muscle of a mammal; and
nitinol elements included in or on the support material,
wherein:
the nitinol elements are configured to be set into an austenite super-elastic phase by a body temperature of the mammal when the support material is wrapped around the joint, ligament, tendon, or muscle, and
the nitinol elements are located and configured to provide a counteracting force to a force exerted on the joint, tendon, ligament, or muscle when the joint, tendon, ligament, or muscle is bent beyond a predetermined angle of extension of the joint, tendon, ligament, or muscle.

2. The support device of claim 1, wherein the support material is configured to wrap around an ankle joint of the mammal.

3. The support device of claim 1, wherein the mammal is a human, and the support material is configured to wrap around a toe joint of the human.

4. The support device of claim 1, wherein the support material is configured to wrap around a knee joint of the mammal.

5. The support device of claim 1, wherein the mammal is a human, and the support material is configured to wrap around a hip joint of the human.

6. The support device of claim 1, wherein the mammal is a human, and the support material is configured to wrap around a back and spine of the human.

7. The support device of claim 1, wherein the mammal is a human, and the support material is configured to wrap around shoulders of the human.

8. The support device of claim 1, wherein the mammal is a human, and the support material is configured to wrap around an elbow of the human.

9. The support device of claim 1, wherein the mammal is a human, and the support material is configured to wrap around a wrist joint of the human.

10. The support device of claim 1, wherein the mammal is a human, and the support material is configured to wrap around a finger joint of the human.

11. The support device of claim 1, wherein the mammal is a horse.

12. The support device of claim 1, wherein the mammal is a dog.

13. The support device of claim 1, wherein the nitinol elements comprise a plurality of nitinol wires arranged in parallel to one another in a direction to provide the counteracting force.

14. The support device of claim 1, wherein the nitinol elements comprise a plurality of nitinol hollow tubes arranged in parallel to one another in a direction to provide the counteracting force.

15. The support device of claim 1, wherein the nitinol elements comprise a plurality of nitinol wires arranged to intersect one another in directions to provide a grid to provide the counteracting force.

16. The support device of claim 1, wherein the nitinol elements comprise a plurality of nitinol hollow tubes arranged to intersect one another in directions to provide a grid to provide the counteracting force.

17. The support device of claim 1, wherein the nitinol elements comprise patterns in a sheet of nitinol material, the patterns being arranged so that the sheet of nitinol material provides the counteracting force.

18. The support device of claim 17, wherein the patterns are laser cut from the sheet of nitinol material.

19. The support device of claim 1, wherein the nitinol elements are configured to be set into the austenite super-elastic phase by a body temperature of the mammal by setting a ratio of nickel and titanium in the nitinol.

20. The support device of claim 1, wherein the nitinol elements are configured to provide the counteracting force based on diameters or thickness gage of the nitinol elements.

21. The support device of claim 16, wherein the nitinol elements are configured to provide the grid in a wiring or woven pattern to provide the counteracting force.

22. The support device of claim 1, wherein the support material is an item of clothing.

23. The support device of claim 1, wherein the predetermined angle is an angle beyond a predetermined angle of extension of the joint.

24. A method of mitigating and/or preventing injury, comprising:
wrapping a support material, having nitinol elements incorporated therein or thereon, around a predetermined joint, tendon, ligament or muscle of a mammal; and
wherein:
the nitinol elements are configured to be set into an austenite super-elastic phase by a body temperature of the mammal when the support material is wrapped around the joint, tendon, ligament, or muscle, and
the nitinol elements are located and configured to provide instantaneous dynamic counteracting force(s) to a force(s) exerted on the joint, tendon, ligament, or muscle when the joint, tendon, ligament, or muscle is bent beyond a predetermined angle of extension of the joint to minimize or prevent injury, and
the nitinol elements are located and configured to provide continuous dynamic support counteracting force(s) to a force(s) exerted on the joint, tendon, ligament, or muscle when the joint, tendon, ligament, or muscle is bent up to a predetermined angle of extension of the joint acting continuously to support and reduce fatigue to minimize acute and chronic injury.

25. A method of optimizing performance of an exoskeleton device for mitigating and/or preventing injury to a user wearing the exoskeleton device, comprising:
arranging nitinol elements comprising at least one of nitinol wires, nitinol tubes and nitinol sheets in and/or on the exoskeleton device in a predetermined arrangement utilizing a design tool configured to perform at least one of finite element analysis, artificial intelligence analysis, and modeling and simulation analysis to integrate data, stored in a database accessible to the design tool, regarding at least one of human and/or animal anatomy, kinesiology, and ergonomics.

26. The method of claim 25, wherein the arranging of the nitinol elements includes selection of a plurality of the nitinol elements and characteristics of the nitinol including at least on of: element type, diameter, thickness, gage, number, length, shapes, nickel: titanium ratio, placement, and arrangement of the nitinol elements.
